(19) United States
(12) Patent Application Publication
(10) Pub. No.: US 2016/0068459 A1
(43) Pub. Date: Mar. 10, 2016

(54) IMMOBILIZED CATALYTICALLY ACTIVE COMPOSITION FOR HYDROFORMYLATION OF OLEFIN-CONTAINING MIXTURES

(71) Applicant: EVONIK DEGUSSA GMBH, Essen (DE)

(72) Inventors: Katrin Marie DYBALLA, Recklinghausen (DE); Robert FRANKE, Marl (DE); Hanna HAHN, Duisburg-baerl (DE); Marc BECKER, Dortmund (DE); Andreas SCHOENWEIZ, Erlangen (DE); Jonas DEBUSCHEWITZ, Erlangen (DE); Simon WALTER, Erlangen (DE); Rene WOELFEL, Erlangen (DE); Marco HAUMANN, Velden (DE); Peter WASSERSCHEID, Erlangen (DE); Andre KAFTAN, Fuerth (DE); Mathias LAURIN, Nuemberg (DE); Joerg LIBUDA, Neunkirchen am Brand (DE)

(73) Assignee: Evonik Degussa GmbH, Essen (DE)

(21) Appl. No.: 14/785,513

(22) PCT Filed: Apr. 16, 2014
(86) PCT No.: PCT/EP14/57793

§ 371 Date: Oct. 19, 2015

(30) Foreign Application Priority Data
Apr. 19, 2013 (DE) 10 2013 207 104.3
Dec. 6, 2013 (EP) PCT/EP2013/075833
Apr. 15, 2014 (DE) 10 2014 207 246.8

(51) Int. Cl. C07C 45/50; B01J 31/18; B01J 31/16; B01J 31/24

(52) U.S. Cl. CPC C07C 45/505; B01J 31/2457; B01J 31/185; B01J 31/1616; B01J 2231/321; B01J 2531/822

(57) ABSTRACT

A composition and the use of said composition as catalytically active composition in processes for synthesis of chemical compounds, especially the hydroformylation of olefinically unsaturated hydrocarbon mixtures.

The present invention provides a composition and the use of said composition as catalytically active composition in processes for synthesis of chemical compounds, especially the hydroformylation of olefinically unsaturated hydrocarbon mixtures.

The reactions between olefin compounds, carbon monoxide and hydrogen in the presence of a catalyst to give the aldehydes with one carbon atom more are known as hydroformylation or the oxo process (Scheme 1). The catalysts used in these reactions are frequently compounds of the transition metals of group VIII of the Periodic Table of the Elements, especially rhodium or cobalt catalysts. Known ligands include, for example, compounds of the phosphine, phosphite and phosphonite classes each comprising trivalent phosphorus P^{III}. A good overview of the state of the hydroformylation of olefins can be found in B. CORNILS, W. A. HERRMANN, “Applied Homogeneous Catalysis with Organometallic Compounds”, vol. 1 & 2, VCH, Weinheim, N.Y., 1996 or R. Franke, D. Selent, A. Börner, “Applied Hydroformylation”, Chem. Rev., 2012, DOI: 10.1021/cr3001803.

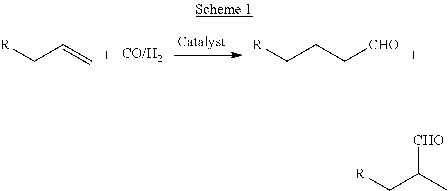

Aldehydes, especially linear aldehydes such as butyraldehyde, valeraldehyde, hexanal and octanal, are of industrial significance as starting materials for plasticizer alcohols, surfactants and fine chemicals.

In 2008, a total of more than 8 million tonnes of oxo process products were produced by means of hydroformylation.

Catalysts which are generally used in the context of the hydroformylation reaction are especially rhodium and cobalt compounds in the presence of ligands. Catalysts used nowadays in the hydroformylation processes are particularly homogeneously dissolved rhodium-based organometallic catalysts, since it is possible here to choose much milder reaction conditions by contrast with the cobalt-based processes (see: H.-W. Bohnen, B. Cornils, Adv. Catal. 2002, 47, 1).

The hydroformylation of olefins using rhodium-comprising catalyst systems is conducted essentially according to two basic variants.

In one, the Ruhrchemie/Rhone-Poulenc process, the catalyst system consisting of rhodium and a water-soluble ligand, usually alkali metal salts of sulfonated phosphines, is dissolved in an aqueous phase. The reactant-product mixture forms a second liquid phase. There is a flow of synthesis gas and olefin, if it is gaseous, through the two phases which are mixed by stirring. The reactant-product mixture is separated from the catalyst system by phase separation. The organic phase removed is worked up by distillation (see: C. W. Kohlpaintner, R. W. Fischer, B. Cornils, Appl. Catal. A Chem. 2001, 221, 219).

Disadvantages of this process, in addition to the high capital investment and the high operating costs, are that it is only possible to use water-stable ligands and that rhodium losses resulting from leaching are unavoidable. This is particularly problematic since specifically rhodium compounds are comparatively costly noble metal complexes, since rhodium is one of the costliest metals in existence.

In the other variant, the catalyst system comprising rhodium is homogeneously dissolved in an organic phase. Synthesis gas and input olefin are introduced into this phase. The reaction mixture drawn off from the reactor is separated by distillation or membrane separation, for example, into a product-reactant phase and a high boiler phase containing the rhodium-comprising catalyst system. The phase containing the rhodium-comprising catalyst system is recycled into the reactor; the other phase is worked up by distillation (see: K.-D. Wiese, D. Obst, Hydroformylation in: Catalytic Carbonylation Reactions; M. Beller (Ed.), Topics in Organometallic Chemistry 18, Springer, Heidelberg, Germany, 2006, 1).

The hydroformylation gives rise to high boilers. For the most part, these are aldol addition or aldol condensation products of the aldehydes formed. In order that the high boiler concentration in the reactor remains limited, a substream, ideally one in which the high boilers are concentrated, has to be discharged. Rhodium compounds are present in this substream. In order to keep the rhodium losses small, rhodium has to be recovered from this discharge stream. The separation of rhodium from such streams is incomplete and complex. Further rhodium losses occur as a result of rhodium cluster formation. These rhodium clusters separate out on equipment walls and may form alloys with the equipment materials. These amounts of rhodium are no longer catalytically active and, even after the plant has been shut down, can be recovered only in a very complex manner and also only in part.

Since the economic viability of an industrial hydroformylation process is substantially dependent on the specific rhodium consumption because of the exceptionally high cost of rhodium in the last few years, attempts have been made to develop alternative processes which feature lower specific rhodium losses.

The starting point in the development of novel hydroformylation processes was the idea of immobilizing rhodium-containing catalyst systems which were previously in homogeneous form in the reaction mixture. This may be referred to as the heterogenization of a reaction conducted homogeneously in principle—in this case the hydroformylation reaction.

In the last few decades, numerous techniques for the immobilization of homogeneous catalysts have been developed, and many of these concepts have been employed for hydroformylation reactions (see: M. Beller, B. Cornils, C. D. Frohning, C. W. Kohlpaintner, J. Mol. Catal. 1995, 104, 17).

The heterogenization of the catalyst complexes by immobilization on porous support materials has been studied in detail. Such heterogenization can be achieved, for example, by covalent anchoring of the rhodium complex on the support via spacer ligands (see: V. A. Likholobov, B. L. Moroz, Hydroformylation on Solid Catalysts in: Handbook of Heterogeneous Catalysis, 2nd ed.; G. Ertl, H. Knoezinger, F. Schüith, J. Weitkamp (eds.), Wiley-VCH, Weinheim, Germany, 2008, 3663).

Aside from the supported aqueous phase (SAP) concept (see H. Delmas, U. Jaeuregui-Haza, A.-M. Wilhelm, Supported Aqueous-Phase Catalysis as the Alternative Method in: Multiphase Homogeneous Catalysis, B. Cornils, W. A. Herrmann, I. T. Horváth, W. Leitner, S. Mecking, H. Olivier-Bourbigou, D. Vogt (Eds.), Wiley-VCH, Weinheim, Germany, 2005), which, however, is unsuitable for hydrolysis-sensitive ligands, the supported liquid phase (SLP) concept is a further concept for heterogenization of homogeneous catalyst complexes. This involves applying a liquid catalyst solution to a porous support material. This concept has already been known for more than 40 years (see: P. Rony, J. Catal. 1969, 14, 142; G. J . K. Acres, G. C. Bond, B. J. Cooper, J. A. Dawson, J. Catal. 1969, 6, 139). Liquid phases used for the hydroformylation include molten salts, for example triphenylphosphine (TPP). TPP serves here as solvent for the catalyst complex, but also as ligand, and is therefore used in a large excess. A problem with such a great ligand excess in catalyst systems under consideration is the formation of various transition metal complexes which can result in suppression of catalytic activity.

The most promising development to date is the hydroformylation of olefins to afford aldehydes using what are known as supported ionic liquid phase catalyst systems or SILP catalyst systems for short.

These are catalytically active compositions in a multiphase system which consist of a solid inert porous support material enveloped in an ionic liquid—called the SILP phase—with the transition metal-comprising, especially rhodium-comprising, catalyst present therein.

### See:

M. Naumann, K. Dentler, J. Joni, A. Riisager, P. Wasserscheid, Adv. Synth. Catal. 2007, 349, 425;
S. Shylesh, D. Hanna, S. Werner, A. T. Bell, ACS Catal. 2012, 2, 487;
M. Jakuttis, A. Schoenweiz, S. Werner, R. Franke, K.-D. Wiese, M. Haumann, P. Wasserscheid, Angew. Chem. Int. Ed. 2011, 50, 4492.

A. Riisager, P. Wasserscheid, R. van Hal, R. Fehrmann, J. Catal. 2003, 219, 252 describes the hydroformylation of propene under SILP conditions, and without the addition of an ionic liquid. The reaction was conducted at 100° C. for 5 hours. In this context, the experiments without IL were notable for an elevated activity. The authors assume that, in this case, the majority of the active complex is adsorbed at the surface, which is not subject to any mass transfer limitation, unlike the experiments which were conducted in the presence of IL.

With SILP catalyst systems, it is possible to combine the advantages of homogeneously and heterogeneously catalysed synthesis reactions. This relates particularly to the product removal and recycling of the catalyst, especially of the transition metals present therein, which is found to be difficult and inconvenient in the case of homogeneously conducted synthesis reactions. Heterogeneously catalysed synthesis reactions, in contrast, may be limited by heat and mass transfer, which reduces the activity of the solid catalyst system; lower chemo- and stereoselectivities are also observed in heterogeneously catalysed synthesis reactions.

The use of a highly active and selective catalyst system is not the only important factor for the economic operation of a continuous process for hydroformylation. Particular aspects that play a crucial role are catalyst recycling—associated with the product removal—and ligand stability—not only in view of the high costs of rhodium and ligands, but also of the only partially understood influence of impurities from ligand degradation processes on the activity and product spectrum.

A disadvantage of the SILP process described in this context is the use of the ionic liquid, called IL for short; the long-term toxicity of these ionic liquids is not entirely clear as yet, and it has been found that some possible cations and anions are toxic to the environment. One example is comparatively long alkyl chains, which are toxic to the aquatic environment. Two further problems are that production costs are still too high and that many ionic liquids lack stability to comparatively high temperatures.

Ionic liquids consist exclusively of ions (anions and cations). In principle, ionic liquids are salt melts having low melting points. In general, these are considered to include not just the salt compounds that are liquid at ambient temperature but also all of those that melt below 100° C. In contrast to conventional inorganic salts such as sodium chloride (melting point 808° C.), lattice energy and symmetry are reduced through delocalization of charge in ionic liquids, which can lead to melting points down to below −80° C. (Römpp's Chemical Dictionary). They are differentiated from salt melts in that ionic liquids normally contain organic cations rather than inorganic cations (P. Wasserscheid & T. Welton, Ionic Liquids in Synthesis, Volume 1, 2nd edition, Wiley-VCH, Weinheim, 2008).

An additional factor is that the commercially available ILs, because of their synthesis, may contain traces or even comparatively large amounts of water in most cases. The drying of these ionic liquids is generally very complex and problematic, since it does not succeed in all cases.

The additional introduction of water via the ionic liquid is particularly critical, since it is common knowledge that organophosphorus ligands in hydroformylation are subject to an inherent degradation and deactivation process [P. W. N. M. van Leeuwen, in Rhodium Catalyzed Hydroformylation, P. W. N. M. van Leeuwen, C. Claver (eds.), Kluwer, Dordrecht, 2000].

Side reactions and degradation reactions may, for example, be hydrolysis, alcoholysis, transesterification, Arbuzov rearrangement, P—O bond cleavage and P—C bond cleavage [P. W. N. M. van Leeuwen, in Rhodium Catalyzed Hydroformylation, P. W. N. M. van Leeuwen, C. Claver (eds.), Kluwer, Dordrecht, 2000; F. Ramirez, S. B. Bhatia, C. P. Smith, Tetrahedron 1967, 23, 2067-2080; E. Billig, A. G. Abatjoglou, D. R. Bryant, R. E. Murray, J. M. Maher, (Union Carbide Corporation), U.S. Pat. No. 4,789,753 1988; M. Takai, I. Nakajima, T. Tsukahara, Y. Tanaka, H. Urata, A. Nakanishi, EP 1 008 581 B1 2004.].

The effect of ligand deactivation and degradation is that less active ligand is present in the system, which can have an adverse effect on the performance of the catalyst (conversion, yield, selectivity).

Thus, additional introduction of substances that accelerate this catalyst degradation, for example introduction of water via the IL, should be avoided.

According to the literature, however, purely heterogeneous catalysts suffer from a low hydroformylation activity but feature quite a high hydrogenation activity which is undesirable in this case (see: a) M. E. Davis, E. Rode, D. Taylor, B. E. Hanson, J. Catal.1984, 86, 67; b) S. Naito, M. Tanimoto, J. Chem. Soc. Chem. Commun. 1989, 1403; c) G. Srinivas, S. S. C. Chung, J. Catal. 1993, 144, 131). Without the presence of a liquid reaction phase in which the organometallic catalyst complex is present in dissolved form, poor regioselectivity is often found.

It is an object of the present invention to develop a process which both enables a favorable catalyst removal by using a catalytically active composition including one or more catalyst complexes on a heterogeneous support and dispenses with the addition of further components. Thus, for example, the addition of an IL as envisaged by the SILP process was to become superfluous. In this way, it is firstly possible to save costs for synthesis of the IL or the procurement thereof; secondly, it is possible to avoid the introduction of catalyst poisons such as water via the IL.

It is a further object of the present invention to develop a process which enables both favorable catalyst removal and shortening of the dynamic process of pore filling and the establishment of the steady equilibrium state between condensation and evaporation.

This object is achieved by a composition as claimed in claim 1.

A composition comprising:

a) at least one inert porous support material;
b) at least one metal selected from the eighth transition group of the Periodic Table of the Elements;
c) at least one organic phosphorus compound;
d) at least one high-boiling liquid on the inert porous support material, having a lower vapor pressure than 0.074 MPa at 100° C. and 1.0 MPa,
wherein the composition is free of ionic liquids.

In the development of the process, simulation tools based on a thermodynamic model are used. In this case, the NRTL-RK physical data method is used. This is an activity coefficient model (g^{E} model) for description of the liquid phase. The vapor phase is described by a state equation, in this case by the Redlich-Kwong state equation which gives a good description of the vapor phase up to moderate pressures. The behavior of multicomponent systems is calculated in advance in the NRTL model from information from the binary systems. The vapor pressure curves of the pure substances are calculated by means of the extended Antoine equation. The parameters which have been fitted to measurement data were adopted for the purpose from AspenPlus© Version 7.3.

In one embodiment, the inert porous support material has the following material properties:

i) mean pore diameter within a range from 1 to 430 nm;
ii) pore volume within a range from 0.1 to 2 ml/g;
iii) BET surface area within a range from 10 to 2050 m²/g.

In one embodiment, the organic phosphorus compounds are selected from phosphines, phosphites and phosphoramidites.

In one embodiment, the phosphines are selected from:

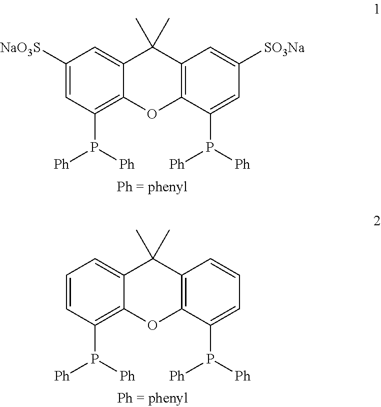

and the phosphites are selected from:

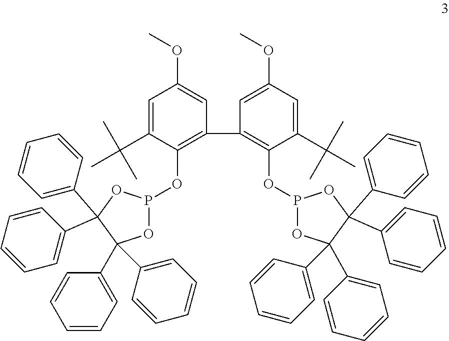 [SMILES: COc1cc(-c2cc(OC)cc(C(C)(C)C)c2OP2OC(c3ccccc3)(c3ccccc3)C(c3ccccc3)(c3ccccc3)O2)c(OP2OC(c3ccccc3)(c3ccccc3)C(c3ccccc3)(c3ccccc3)O2)c(C(C)(C)C)c1]

In one embodiment, the high-boiling liquid is formed in situ during use in a process for chemical synthesis.

In one embodiment, the high-boiling liquid is formed in situ during the hydroformylation of olefin-containing hydrocarbon mixtures.

As well as the composition, a process is also claimed.

A process for hydroformylating olefin-containing hydrocarbon mixtures, comprising the process steps of:

a) introducing an olefin-containing hydrocarbon mixture into a reaction mixture,
b) introducing an inert porous support material into the reaction mixture,
c) introducing a metal selected from cobalt, rhodium, iridium and ruthenium into the reaction mixture,
d) introducing an organic phosphorus compound selected from phosphines, phosphites and phosphoramidites into the reaction mixture,
e) introducing an aldol compound which is not part of the olefin-containing hydrocarbon mixture into the reaction mixture,
f) feeding in H₂ and CO,
g) heating the reaction mixture, with conversion of the olefin to an aldehyde.

The sequence of process steps a) to e) is arbitrary.

In one variant of the process, the aldol compound in process step e) is selected from: 2-methylpent-2-enal (conversion product of ethene hydroformylation, CAS 623-36-9), 2-ethylhex-2-enal (conversion product of propene hydroformylation, CAS 645-62-5), 2-propylhept-2-enal (conversion product of butene hydroformylation, CAS 34880-43-8).

In one variant of the process, the organic phosphorus compound from process step d) is selected from:

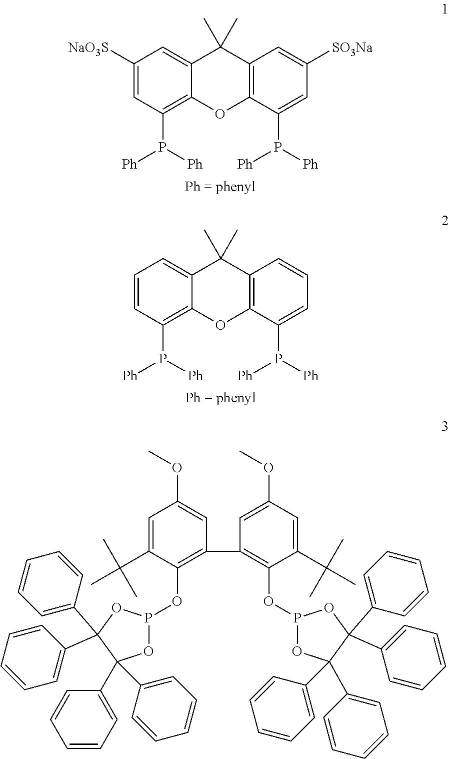

In one variant of the process, the inert porous support material in process step b) is selected from:

silicon dioxide, aluminum oxide, titanium dioxide, zirconium dioxide, silicon carbide, charcoal, mixtures of these components.

In one variant of the process, the inert porous support material in process step b) has the following material properties:

i) mean pore diameter within a range from 1 to 430 nm;
ii) pore volume within a range from 0.1 to 2 ml/g;
iii) BET surface area within a range from 10 to 2050 m²/g.

In one variant of the process, the olefin-containing hydrocarbon mixture is selected from the group comprising:
ethene;
propene;
C4 olefins, C4 paraffins, polyunsaturated compounds.

In one variant of the process, the reaction mixture is free of ionic liquids.

In one variant of the process, the metal, the organic phosphorus compound and the aldol compound are first mixed in a separate vessel before they are introduced into the reaction vessel.

“Reaction vessel” is understood to mean the vessel in which the hydroformylation takes place. This may, for example, be a reactor.

In one variant of the process, the inert porous support material is also added to the mixture before the mixture is introduced into the reaction vessel.

As well as the above-specified calculation/simulation method, it is also possible to calculate the vapor pressure by means of the equation that follows.

With the aid of the Kelvin equation, it is possible to estimate the vapor pressure of liquids under specific reaction conditions in pores of a support (see equation 1). The Kelvin equation describes the change in the vapor pressure of a pure substance at a curved gas/liquid interface with respect to a saturation vapor pressure of an uncurved surface, the starting point according to the definition being an incompressible liquid and an ideal gas as gas phase.

$$p = {p}_{s} \cdot exp ( \frac{- 2 \cdot σ \cdot M}{RT \cdot {δ}_{l}} \cdot \frac{1}{{r}_{Pore}} ) \qquad Equation 1$$

p in equation 1 describes the saturation vapor pressure over a curved liquid surface, pₛ the saturation vapor pressure over an uncurved surface, σ the interfacial tension, M the molar mass, R the universal gas constant, T the temperature, δ the density of the liquid and rₚₒᵣₑ the radius of the pores.

The interfacial tension σ can be calculated via an empirically determined formula according to Brock and Bird (B. E. Poling, J. M. Prausnitz, J. P. O'Connell, Surface Tension in: The Properties of Gases and Liquids, McGraw-Hill, USA, 2001, 691), based on the critical parameter of the liquid (see equations 2 and 3).

$$\begin{matrix}σ = {( {p}_{c} )}^{2 / 3} \cdot {( {T}_{c} )}^{1 / 3} \cdot {Q ( 1 - \frac{T}{{T}_{c}} )}^{1 \; 1 / 9} \\ where & Equation 2 \\ Q = 0.1196 [ 1 + \frac{\frac{{T}_{SP}}{{T}_{c}} \cdot ln ( \frac{{p}_{c}}{1.01325 bar} )}{1 - \frac{{T}_{SP}}{{T}_{c}}} ] - 0.279 & Equation 3\end{matrix}$$

p_{c} and T_{c} describe the critical pressure and temperature; T_{SP} describes the boiling point of the liquid phase.

The invention is illustrated in detail hereinafter by working examples and figures.

### FIGURES

FIG. 1:

Time-resolved operando DRIFTS spectra of the CO vibration regions at selected times between 30 min and 96 h: (a) range between 1950-2200 cm⁻¹, (b) range between 1600-1800 cm⁻¹; and (c) profile of the signal intensities from (a) against time; and (d) profile of the signal intensities from (b) against time. IR signals were recorded daily for about 16 h over an entire experiment run time of 110 h. In the empty regions of (c) and (d), the catalytic reaction is still taking place, but could not be measured throughout because of apparatus restrictions.

FIG. 2:

DRIFTS spectra obtained (i) operando after reaction for 96 h; (ii) for silica 100 impregnated with pure aldol (E)-2-ethylhex-2-enal; (iii) for silica 100 impregnated with pure isobutanal; and (iv) for silica 100 impregnated with pure n-butanal.

FIG. 3:

Comparison of conversion (♦ and ⋄) and n/iso selectivity (▪ and □) in the gas phase hydroformylation of propene with an identical catalyst in a conventional tubular reactor (open symbols: ⋄, □) or operando IR reactor (closed symbols: ♦, ▪). Parameters: m_{cat}=700 mg (for operando IR reactor: 60 mg), m_{Rh}=0.2% by weight, ligand/Rh=5 with ligand 3, T=80° C., p=0.2 MPa, pₚᵣₒₚₑₙₑ=0.04 MPa, p_{H2}=p co=0.08 MPa, residence time=6 s.

FIG. 4:

Conversion-time diagram for ethene hydroformylation with Rh-1 catalysts on Trisopor 423 with (♦) and without (⋄) initial addition of 2-methyl-2-pentenal. Parameters: m_{cat}=2.30 g (⋄) or 2.36 g (♦), m_{Rh}=0.2% by weight, ligand/Rh=5, m_{2-methyl-2-pentenal}=2.6% by weight, T=353.15K, p=2.0 MPa, pₑₜₕₑₙₑ=0.1 MPa, p_{H2}=p_{co}=0.95 MPa, residence time=30 s.

FIG. 5:

Headspace GC/MS analysis of Rh-1 catalysts on Trisopor 423 with ( - - - ) and without (-) initial addition of 2-methyl-2-pentenal after use in ethene hydroformylation over 70 h. The main signals stem from 2-methyl-2-pentenal, 2-methyl-2-pentanal and propanal.

FIG. 6:

Conversion-time diagram for 1-butene hydroformylation with Rh-2 catalysts on an activated carbon support with (♦) and without (⋄) initial addition of 2-propyl-2-heptenal and the n/iso selectivity with (▪) and without (≡) initial addition of 2-propyl-2-heptenal. Parameters: m_{cat}=2.30 g (⋄,□) or 2.42 g (♦,▪), m_{Rh}=0.2% by weight, ligand/Rh=5, m_{2-propyl-2-heptenal}=5.2% by weight, T=373.15K, p=1 MPa, p_{1-butene}=0.18 MPa, p_{H2}=p_{co}=0.41 MPa, residence time=20 s.

FIG. 7:

Schematic diagram of the model for pore filling

### DETERMINATION OF VAPOR PRESSURE

In the development of the process, simulation tools based on a thermodynamic model are used. In this case, the NRTL-RK physical data method is used. This is an activity coefficient model (g^{E} model) for description of the liquid phase. The vapor phase is described by a state equation, in this case by the Redlich-Kwong state equation which gives a good description of the vapor phase up to moderate pressures. The behavior of multicomponent systems is calculated in advance in the NRTL model from information from the binary systems. The vapor pressure curves of the pure substances are calculated by means of the extended Antoine equation. The parameters which have been fitted to measurement data were adopted for the purpose from AspenPlus© Version 7.3.

TABLE 1
Vapor pressure as a function of the temperature
of aldol compound in process step e).

| | Vapor pressure/ | Vapor pressure/ | Vapor pressure/ |
|---|---|---|---|
| Tempera- | MPa^{[a]} | MPa^{[b]} | MPa^{[c]} |
| ture/K | 2-Methylpent-2-enal | 2-Ethylhex-2-enal | 2-Propylhept-2-enal |
| 293.15 | 0.00084 | 0.00013 | |
| 298.15 | 0.00113 | 0.00019 | |
| 303.15 | 0.00151 | 0.00026 | |
| 308.15 | 0.00199 | 0.00036 | |
| 313.15 | 0.00259 | 0.00049 | |
| 318.15 | 0.00334 | 0.00067 | |
| 323.15 | 0.00427 | 0.00088 | |
| 328.15 | 0.00542 | 0.00116 | |
| 333.15 | 0.00681 | 0.00152 | |
| 338.15 | 0.00849 | 0.00196 | |
| 343.15 | 0.01051 | 0.00250 | |
| 348.15 | 0.01292 | 0.00318 | |
| 353.15 | 0.01577 | 0.00400 | 0.00122 |
| 358.15 | 0.01912 | 0.00499 | 0.00153 |
| 363.15 | 0.02304 | 0.00618 | 0.00190 |
| 368.15 | 0.02760 | 0.00760 | 0.00236 |
| 373.15 | 0.03287 | 0.00929 | 0.00291 |
| 378.15 | 0.03893 | 0.01129 | 0.00357 |
| 383.15 | 0.04587 | 0.01362 | 0.00437 |
| 388.15 | 0.05378 | 0.01634 | 0.00532 |
| 393.15 | 0.06274 | 0.01950 | 0.00645 |
| 398.15 | 0.07286 | 0.02315 | 0.00779 |
| 403.15 | 0.08424 | 0.02733 | 0.00938 |
| 408.15 | 0.09699 | 0.03212 | 0.01124 |
| 413.15 | 0.11121 | 0.03757 | 0.01343 |
| 418.15 | 0.12702 | 0.04374 | 0.01599 |
| 423.15 | 0.14453 | 0.05071 | 0.01897 |
| 428.15 | 0.16387 | 0.05856 | 0.02243 |
| 433.15 | 0.18517 | 0.06735 | 0.02644 |
| 438.15 | 0.20853 | 0.07716 | 0.03106 |
| 443.15 | 0.23410 | 0.08809 | 0.03639 |
| 448.15 | 0.26201 | 0.10022 | 0.04251 |
| 453.15 | 0.29239 | 0.11363 | 0.04951 |
| 458.15 | 0.32537 | 0.12844 | |
| 463.15 | 0.36110 | 0.14472 | |
| 468.15 | 0.39972 | 0.16259 | |
| 473.15 | 0.44137 | 0.18215 | |

^{[a]}Measurement data from Eidus, Ya. T.; Lapidus, A. L., Hydroformylation of Ethylene with a Mixture of Carbon Monoxide and Hydrogen using Rhodium Catalysts, Pet. Chem. USSR, Volume 7, Issue 1, 1967, Pages 9-15, http://dx.doi.org/10.1016/0031-6458(67)90003-2 and from Auwers, K.; Eisenlohr, F., Spektrochemische Untersuchungen. Über Refraktion und Dispersion von Kohlenwasserstoffen, Aldehyden [Spectroscopic Studies. On Refraction and Dispersion of Hydrocarbons, Aldehydes], Volume 82, Issue 1, pages 65-180, Dec. 17, 1910, http://dx.doi.org/10.1002/prac.19100820107. Vapor pressure curve fitted with AspenPlus © Version 7.3.
^{[b]}Measurement data from Dykyj, J.; Seprakova, M.; Paulech, J., Vapor pressure of two alcohols C8 and of two aldehydes C8, Chem. Zvesti, 15, 1962. Vapor pressure curve fitted with AspenPlus © Version 7.3.
^{[c]}Dechema Detherm ID: PVT-7008.1988.

By way of example, table 2 shows the physicochemical data for n-pentanal (hydroformylation product of a C4 olefin).

TABLE 2
Physicochemical data of n-pentanal and n-decanol
at T = 373.15 K and p = 1.0 MPa. Values
identified by * come from FLUIDAT ® database,
Bronkhorst High-Tech B.V., the Netherlands.

| | | | | | | M/g |
|---|---|---|---|---|---|---|
| Compound | pₛ/MPa | p_{c}/MPa | T_{c}/K | T_{SP}/K | δₗ/kgm⁻³ | mol⁻¹ |
| n-Decanol | 0.0013 * | 2.23 * | 700.0 * | 513.1 * | 790.7 * | 158.3 |
| n-Pentanal | 0.093 * | 3.55 * | 554.0 * | 375.8 * | 685.7 * | 86.1 |

All values identified by * come from the FLUIDAT ® database provided by Bronkhorst High-Tech B.V., the Netherlands. For reaction conditions of 373.15 K and 1.0 MPa, at a mean pore diameter for a porous support material of 6 nm, under given conditions, a vapor pressure of n-pentanal of 0.074 MPa can be calculated. Considering a support material having exclusively micropores (mean pore diameter of 1 nm), the vapor pressure in the pores is reduced to 0.024 MPa.

In order to further determine the conditions under which both the formation and the enrichment of high-boiling liquids proceed in porous networks of inert support materials, as formed, for example, as conversion products from aldehydes, the inert porous support material and the substrate to be hydroformylated, for example olefins or olefinic hydrocarbon mixtures, are varied.

High-boiling compounds can be characterized by estimation according to equation 1 (bearing in mind that equation 1, by definition, does not apply to complex mixtures) or by description via a physical data model (NRTL-RK physical data method, AspenPlus© Version 7.3), as having a lower vapor pressure based on the mean pore diameter of the support material than the aldehyde product formed in the reaction.

The behavior of the catalytically active composition under reaction conditions was examined with the aid of an operando IR reactor in the continuous gas phase hydroformylation of propene. In the course of this, the catalytically active composition was analyzed by means of DRIFTS spectroscopy over the entire experiment run time and, at the same time, the progress of reaction was measured by means of online gas chromatography. This spectroscopic analysis method is presented by A. Drochner and G. H. Vogel in Methods in Physical Chemistry 2012, 445-475, ISBN 9783527327454.

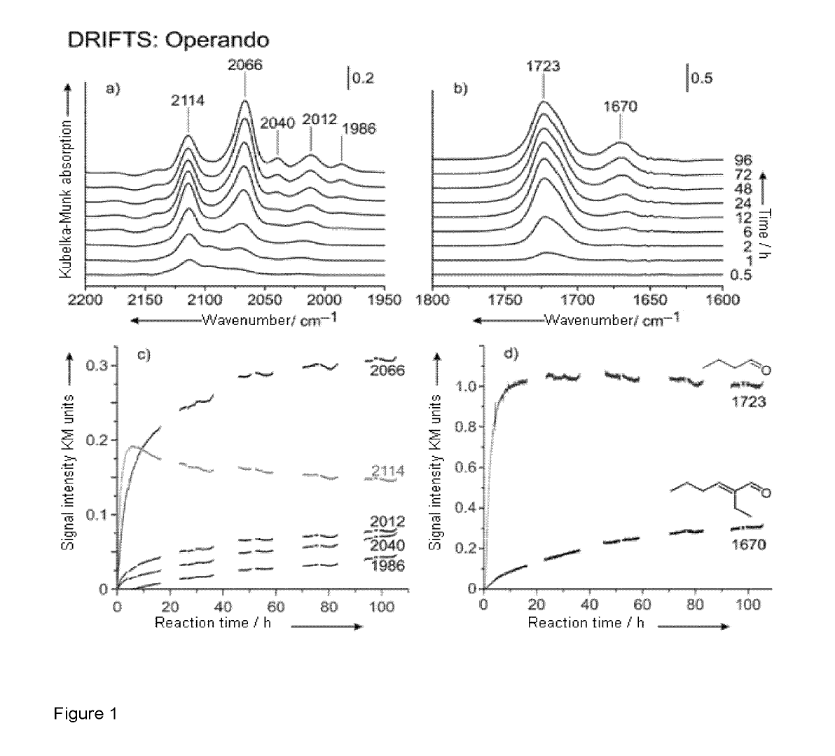

FIG. 1 shows selected spectra in the CO region in the range of (a) 2200 to 1950 cm⁻¹ and (b) of 1800 to 1600 cm⁻¹. It is apparent from FIG. 1a that, even shortly after the admission of CO into the measurement cell, a band at 2114 cm⁻¹ together with a shoulder which extends up to 2050 cm⁻¹ is formed. The maximum intensity of this band has been attained after only 6 h and decreases gradually thereafter (cf. profile of the signal intensities against time in FIG. 1c). The intensities of the bands at 2066, 2040, 2012 and 1986 cm⁻¹, in contrast, increase together. The profile of these four bands against time remains parallel over the entire duration of the experiment. The intensities rise to an enhanced degree during the first 20 h and reach nearly a constant level after more than 60 h of reaction time.

These bands can be assigned to the known (ee) and (ea) enantiomers of the catalytically active species which arise through activation of the catalyst precursor complex under influence of the ligand and synthesis gas, as already disclosed by D. Selent, R. Franke, C. Kubis, A. Spannenberg, W. Baumann, B. Kreidler, A. Börner in Organometallics 2011, 30, 4509. The band at 2114 cm⁻¹ together with the shoulder concurs with infrared data of isolated gem-dicarbonyl Rh(CO)₂ compounds, as already disclosed by S. M. McClure, M. J. Lundwall, D. W. Goodman in Proc. Nat. Acad. Sci. 2011, 108, 931 and M. Frank, R. Kühnemuth, M. Bäumer, H.-J. Freund in Surf. Sci. 2000, 454-456, 968.

This indicates the presence of highly dispersed rhodium, which probably comes from the catalyst preparation. Independently of this, no band was found at 2080 cm⁻¹, which would indicate linear-adsorbed CO and hence would be a sign of the sintering or formation of rhodium particles, as disclosed by J. Evans, B. Hayden, F. Mosselmans, A. Murray in Surf. Sci. 1992, 279, 159 and M. Frank, R. Kühnemuth, M. Bäumer, H.-J. Freund in Surf. Sci. 1999, 427-428, 288.

FIG. 1b shows a significant adsorption band at 1723 cm⁻¹ and a further adsorption band at 1670 cm⁻¹. By reference measurements, it was possible to assign these bands to the C═O stretching vibrations of n-butanal (aldehyde) and 2-ethylhex-2-enal (aldol product). The intensity of the band at 1723 cm⁻¹ rises rapidly and reaches a steady state after only 6 h. The band at 1670 cm⁻¹, in contrast, can only be observed from a reaction time of 6 h and the intensity thereof rises over the entire duration of the experiment (see FIG. 1d).

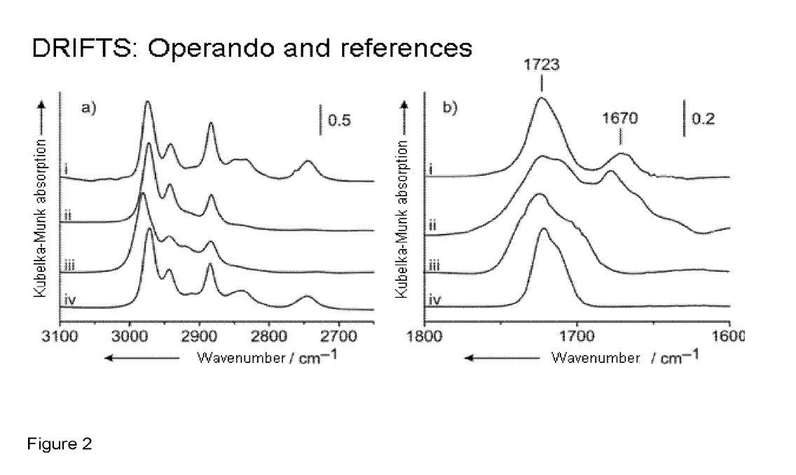

The reference measurements are shown in detail in FIG. 2. Here, the CH and CO stretching vibration ranges for the following systems are compared: (i) a selected spectrum from the abovementioned operando experiment, and (ii-iv) the pure aldehyde and aldol products immobilized on calcined silica 100. In the CH vibration range (see FIG. 2a), the operando spectrum shows the greatest similarity with the spectrum of pure n-butanal (iv). However, the spectra of 2-ethylhex-2-enal (ii) and iso-butanal (iii) do not show any particular features that would enable specific distinction. In the CO vibration range (see FIG. 2b), the intense band at 1723 cm⁻¹ is present in every product. This band is thus attributable principally to the linear aldehyde n-butanal. Conversely, the shoulder observed in the operando spectrum (i) at 1670 cm⁻¹ can be found only in the reference spectrum of 2-ethylhex-2-enal (ii) and hence confirms the presence of the aldol product in the system examined.

In addition to the results mentioned so far, FIG. 1d shows very clearly that, in the first 6 h of the reaction, both the formation rate of butanal and that of the aldol product are at their highest. After the formation of the butanal has stabilized (IR signal reaches saturation), the formation rate of the aldol falls, although a steady state of aldol formation has not been achieved over the entire run time of the experiment. However, it is noticeable that the formation rate falls constantly. It can thus be concluded that the aldehydes are formed at first up to an equilibrium concentration in the pores of the support material, and the aldol products are then formed over the further reaction time. These findings support the model of filled pores in which aldehyde and aldol products form in the pore network of SiO₂-supported Rh catalysts during the continuous hydroformylation reaction and partly condense out. After the reaction has ended, the catalytically active composition was purged with argon (10 mL min⁻¹), whereupon the gas phase signals of the reactants disappear but the characteristic vibration bands of the aldehyde and aldol products remain measurable.

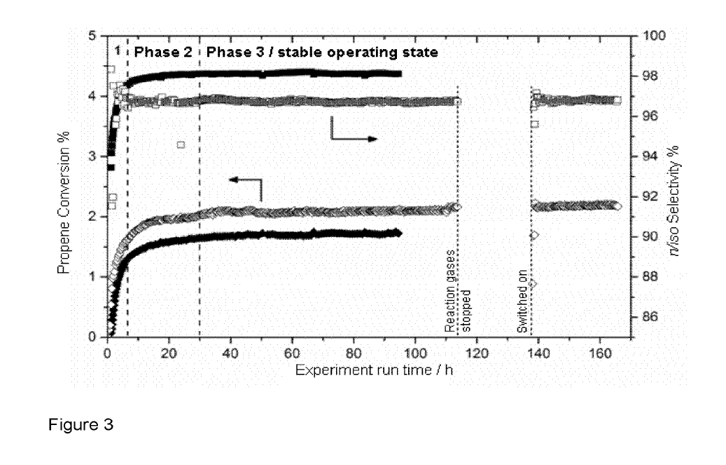

In order to verify the behavior of the catalytically active composition in the operando DRIFTS cell, it was tested under comparable reaction conditions in a conventional hydroformylation system with a tubular reactor as well. The experimental results from the two systems are compared in FIG. 3.

In spite of the different reactor design, the results are very comparable. In both experiments, the propene conversion starts at 0% and rises rapidly within the first 6 h. After an experiment run time of about 30 h, a stable conversion of 1.6% or 2.1% is achieved in the two cases. The selectivity based on linear butanal reaches a constant value of 98% or 97% after about 12 h. The lower n/iso selectivity compared to disclosed studies with Rh-benzpinacol-based complex catalysts having, for example, the bisphosphite 3 as ligand is attributable to the reaction conditions (T=80° C., pₜₒₜₐₗ=0.2 MPa). The catalyst behavior shown can be divided into 3 phases: Within the first 6 h, the catalyst shows clear activation behavior (phase 1) in which conversion and n/iso selectivity change significantly over time. Between 6 h and 30 h of experiment run time (phase 2), the changes are much less marked and the regioselectivity reaches a stable level, although the catalyst activity is still rising slightly. After 30 h, a constant level is reached both for the propene conversion and for the n/iso selectivity, in which there is continuously no further significant change (phase 3).

Evidence that, after the attainment of a stable operating point for the first time, the formation of the condensed aldehyde and aldol phase is complete for defined reaction conditions and there is no further major change in the catalyst composition thereafter is given by the extended experiment in the tubular reactor. After 115 h, the metering of the substrate gases was switched off and the catalyst was stored under helium overnight. After the reaction has been restarted under identical conditions, the system shows the same catalytic behavior as before, except without any marked activation phase.

All the studies conducted are conducted with retention of the protective gas atmosphere (argon).

### EXPERIMENTAL

### Chemicals

(Acetylacetonato)dicarbonylrhodium(I) (Rh(acac)(CO)₂), 9,9-dimethyl-4,5-bis(di-tert-butyl-phosphino)xanthene (xantphos, 2) and dichloromethane (HPLC purity) were purchased from Sigma Aldrich and used without further purification. The sulfoxantphos ligand, 1 was prepared by a literature method by sulfonation of xantphos. The benzpinacol-based bisphosphite 3 was synthesized according to DE 10 2006 058 682 A1. The macroporous silicon dioxide is commercially available in each case as Trisopor® 423 (particle size 100 to 200 μm, BET surface area in the range of 10-30 m²/g, mean pore diameter 423 nm) from VitroBio GmbH or as silica 100, as used, for example, for preparative column chromatography. The activated carbon used is commercially available (particle size 500 μm, BET surface area in the range of 2000-2010 m²/g) and comes from Blücher GmbH. The macroporous silicon dioxide—Trisopor® 423—and silica 100 too were each calcined at 873.15 K for 18 hours prior to use for production of the catalytically active composition. Ethene (99.95%), propene (99.8%), carbon monoxide (99.97%) and hydrogen (99.999%) were sourced from Linde AG. 2-Methyl-2-pentenal (97%) was purchased from Sigma Aldrich. 2-Propyl-2-heptenal was prepared by a literature method by base-catalyzed aldol reaction of freshly purified n-pentanal. The aldol products formed were separated by subsequent distillation in order to achieve a high purity of 2-propyl-2-heptenal.

### Preparation of the Catalytically Active Composition

All the preparations of the catalytically active composition were effected by means of Schlenk methodology under argon (99.999%). Rh(CO)₂(acac) was dissolved in dichloromethane and stirred for 5 min. A five-fold excess of sulfoxantphos 1, xantphos 2 or bisphosphite 3 (molar ligand/rhodium ratio=5) was likewise initially charged in dichloromethane, stirred for 5 min and added to the rhodium precursor solution. After stirring for a further 5 min, the required amount of calcined macroporous silicon dioxide—Trisopor 423—or silica 100 or activated carbon (mass ratio of rhodium/support material=0.2%) was added. The suspension obtained was stirred for 10 min. Dichloromethane was then drawn off under reduced pressure on a rotary evaporator, and the resulting powder was dried under fine vacuum (10 Pa) overnight before being used as a catalytically active composition. In the compositions with aldol doping, 2-methyl-2-pentanal or 2-propyl-2-heptenal was added to the rhodium ligand solution in a defined amount before the particular support material was added. After stirring for 10 min, dichloromethane was removed again on a rotary evaporator, but there was no additional drying under fine vacuum overnight.

### Catalysis Experiments

All the hydroformylation experiments were conducted in a fixed bed reactor. The dry catalyst material was charged into the tubular reactor and fixed with a piece of glass wool on either side. The overall system was purged three times with helium at room temperature and then pressurized with the reaction pressure (helium). If no pressure drop was found within 15 min, the reactor was heated up to reaction temperature. After the particular volume flow rates had been established, the substrates (ethene, CO and H₂) were passed through the reactor. The reactants were metered in via mass flow regulators (sourced from Bronkhorst). In a mixer filled with glass beads, the reactant gas stream was homogenized before it flowed through the tubular reactor, including the catalyst bed, from the top. The reactor consisted of stainless steel (diameter 12 mm, length 500 mm) and had a porous frit for positioning of the catalyst material on the outlet side. By means of an internal thermocouple, it was possible to record the temperature in the catalyst bed. A 7 μm filter downstream of the reactor prevented additional unwanted discharge of catalyst material. The total pressure in the experimental system was regulated by means of an electronic pressure-retaining valve (source: Samson). On the low-pressure side, the product gas stream was divided with the aid of a needle valve, such that only a small proportion of the total stream was passed to the online gas chromatograph (sourced from Agilent, model: 7890A). The greater proportion was passed directly into the waste air outlet. Through a 6-port valve with a 1 mL sample loop, samples of the product gas stream were injected into the gas chromatograph at regular intervals. The data were evaluated by means of the ChemStation software from Agilent.

### Analysis

The product gas composition was analysed with an online gas chromatograph during the experiment run time. The gas chromatograph was equipped with a GS-GasPro capillary column (from Agilent Technologies, length 30 m, internal diameter 0.32 mm) and a flame ionization detector (FID). Analysis parameters set: injector temperature 523.15 K, split ratio 10:1, constant column flow rate of helium 4.5 ml min⁻¹, detector temperature 533.15 K, heating ramp: starting temperature 533.15 K, hold time 2.5 min, heating to 473.15 K at 20 K min⁻¹, hold time 4 min, total time per analysis 10 min.

Headspace GC/MS analyses were conducted on a Varian 450 gas chromatograph with combined Varian 220-MS mass spectrometer. Samples were injected using a Combi PAL GC autosampler (sourced from CTC Analytics) with a heatable gas-tight syringe and heatable agitator. For each measurement, 0.5 g of the catalyst material for analysis was introduced into a headspace vial and heated to 403.15 K for 15 min. With the aid of the preheated syringe (403.15 K), 500 μL gas samples were injected into the GC. The gas mixture was separated using a FactorFour VF-5 ms capillary column (sourced from Varian, length 30 m, internal diameter 0.25 mm). The components separated were ionized by means of electron impact ionization. Analysis parameters set: injector temperature 523.15 K, split ratio 10:1, constant column flow rate of helium 1.0 ml min⁻¹, heating ramp: starting temperature 313.15 K, hold time 3.0 min, heating to 373.15 K at 5 K min⁻¹, hold time 10.5 min, heating to 473.15 K at 10 K min⁻¹, hold time 0.5 min, total time per analysis 36 min.

The experiments for continuous gas phase hydroformylation of short-chain alkenes (C₂-C₄) showed that purely physisorbed rhodium ligand complexes on a high-porosity support material can indeed be active and selective. The n/iso selectivities found (ratio of linear (n) to branched (iso) product) were comparable in all cases with known values from the literature for homogeneously catalyzed liquid phase reactions. In addition, the catalyst systems exhibited only slight deactivation behavior even over extended experiment run times. By weighing and headspace GC/MS analyses of the deinstalled catalyst samples after the reaction, it was found that the mass of the catalyst material distinctly increases because of the catalytic conversion and this increase in weight is unambiguously attributable to high-boiling conversion products (mainly aldol products) which form through side reactions and further reactions of the aldehydes formed as primary products and remain in the pores of the support materials under the reaction conditions selected. Typical conditions under which this phenomena was observed are: T_{reactor}=353.15-393.15 K and pₜₒₜₐₗ=0.5-2 MPa (pₐₗₖₑₙₑ=0.03-0.18 MPa).

Based on these preliminary studies, in one embodiment for preparation of the inventive composition, a defined amount of corresponding high-boiling liquids, for example aldol products, is added during the preparation; called aldol doping. The amount of aldol product added corresponded to the increase in weight which was measured after 70 h of experiment run time for a previously tested catalyst with purely physisorbed rhodium ligand species. In some cases, it was found that the “aldol-doped” (doped=addition of high-boiling liquids during the preparation of the catalytically active composition) catalysts are more active and more selective in terms of the spectrum of high boilers formed, compared to materials without initial addition of aldol. These were the following systems: Rh-sulfoxantphos (SX, 1) on the SiO₂ support Trisopor 423 (Rh-1/Trisopor 423) and Rh-xantphos (X, 2) on an activated carbon support (Rh-2/activated carbon). Each system was tested with and without addition of a certain amount of the corresponding aldol product, 2-methyl-2-pentenal or 2-propyl-2-heptenal, in the continuous gas phase hydroformylation of ethene (Rh-1/Trisopor423) or 1-butene (Rh-2/activated carbon).

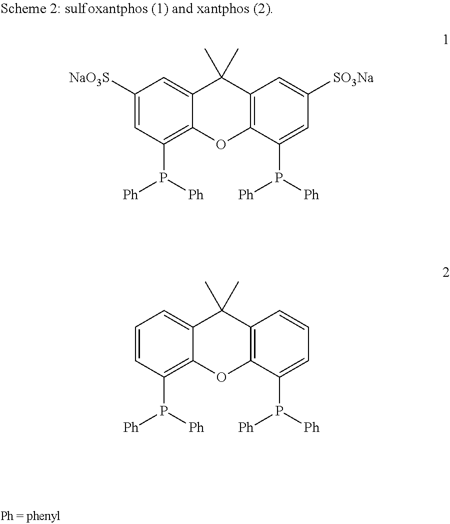

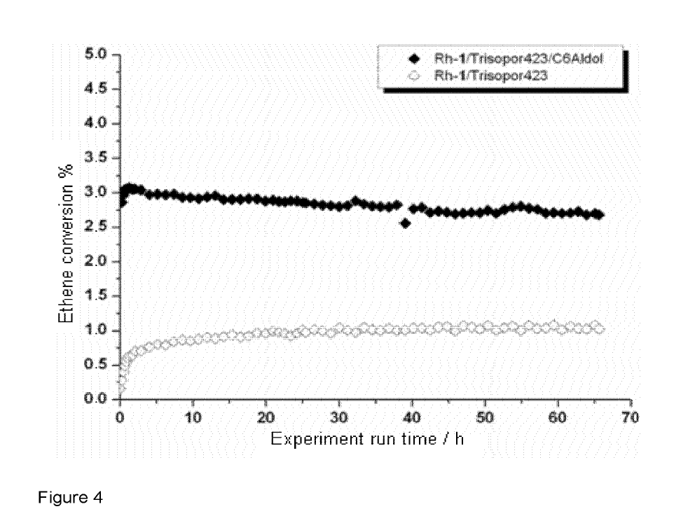

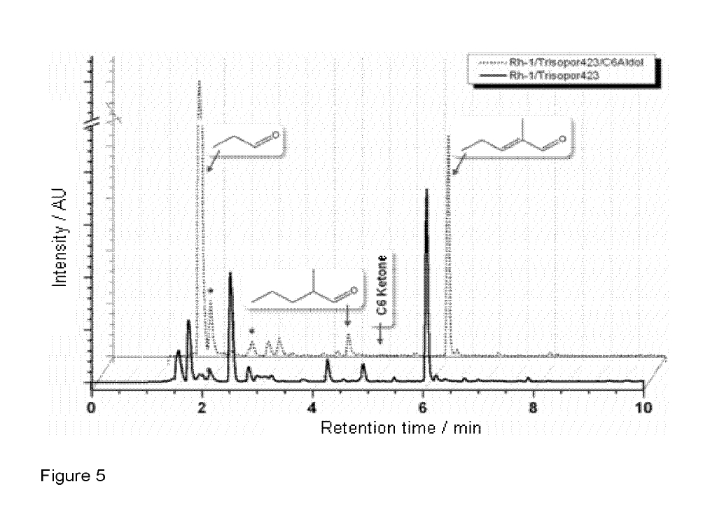

The results for the hydroformylation of ethene with Rh-1/Trisopor423 are shown in FIG. 4. It is clearly apparent that the catalyst without aldol doping has a very marked activation phase which extends nearly over the entire experiment run time of 70 h. The maximum conversion achieved here (Xₘₐₓ) is about 1%. The spent catalyst material was 2.6% by weight heavier after the reaction than that used at the start. By means of headspace GC/MS analysis, it was possible to attribute this increase in weight particularly to the compounds 2-methyl-2-pentenal, 2-methyl-2-pentanal, propanal and a C₆ ketone, which were formed during the catalytic conversion of ethene and remained partly in the pores of the catalyst material (see FIG. 5).

With the aid of headspace GC/MS analysis, it is possible to determine solid compositions, provided that they are at least partly compounds having a sufficiently high vapor pressure. The solid to be examined is introduced into a closed sample vessel and heated under agitation. After a while, there is an equilibrium between the solid sample material and gaseous components. A sample of the gas phase is taken through a septum in the lid of the sample vessel and then analyzed in a GC/MS spectrometer. In the first part (GC), the individual components of the gas sample injected are separated, while these constituents are quantified by mass spectrometry in the second part (MS). With the aid of substance databases and/or reference measurements, the components are assigned qualitatively.

If a catalyst system which has already been laden with 2.6% by weight of pure 2-methyl-2-pentenal during the preparation is used (called aldol doping), in order to immobilize Rh-1 on Trisopor 423, it is possible to achieve a higher maximum conversion under identical reaction conditions (Xₘₐₓ=3.1% based on the ethene used), which remains virtually constant over the experiment run time of 70 h. In addition, the subsequent headspace analysis shows an altered product spectrum with regard to the components formed during the reaction and remaining on the catalyst. Thus, the amount of 2-methyl-2-pentenal is virtually identical, but much more propanal was detected, and less hydrogenation product, 2-methyl-2-pentanal. Integration of the peak areas gives ratios of propanal/2-methyl-2-pentenal and 2-methyl-2-pentanal/2-methyl-2-pentenal of 9.7 and 0.11 respectively. In the case of the purely physisorbed catalyst, i.e. without initial addition of aldol product, the ratios are 0.3 and 0.14 respectively. Moreover, in the case of the “doped” catalyst, the C₆ ketone secondary component was not detected at all. It can thus be stated that controlled aldol doping can firstly increase the activity of the supported catalyst and secondly have a positive effect on by-product formation.

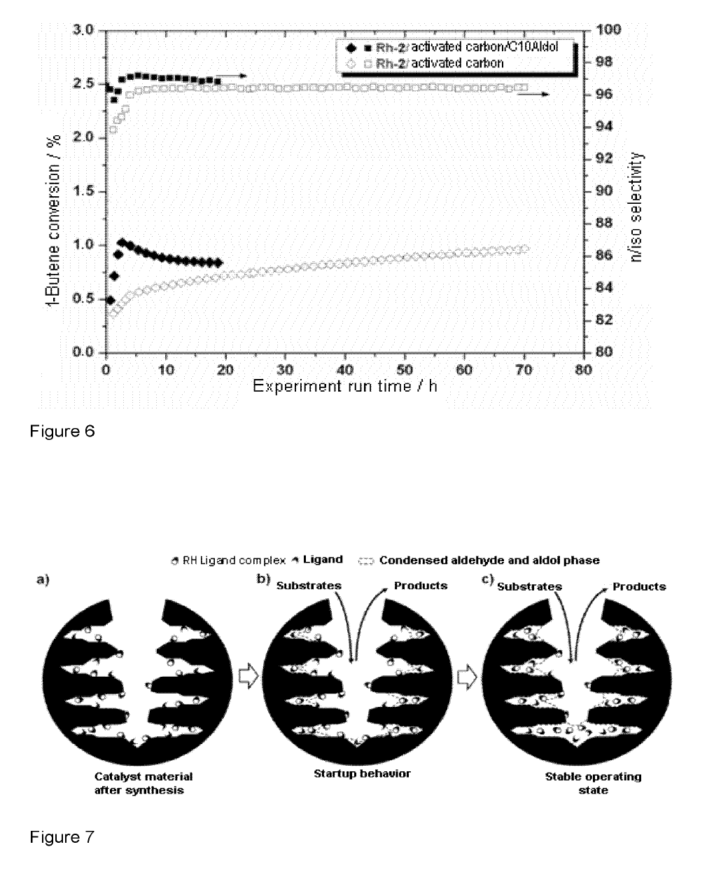

Similar behavior was found for the Rh-2/activated carbon catalyst system which was tested in the hydroformylation of 1-butene. FIG. 6 shows the conversion-time profiles of a catalyst without and with initial addition of 2-propyl-2-heptenal. The amount of C₁₀ aldol product used in this case was 5.2% by weight, which corresponded to the measured increase in weight of the purely physisorbed catalyst system after an experiment run time of 70 h. Here too, it is clearly apparent that the catalyst without aldol doping has marked activation behavior and a maximum conversion of Xₘₐₓ=0.9% is achieved. In contrast, the initial addition of 2-propyl-2-heptenal achieves a slightly higher maximum conversion (Xₘₐₓ=1.1%) established within the first few hours. With increasing experimental duration, a similar conversion is then achieved to that in the case of the undoped catalyst. The regioselectivity for the desired linear pentanal in both cases is about 97%, which corresponds to the values known from the literature in the case of use of Rh-2 species in homogeneous catalysis (for the Rh-2/activated carbon/C10aldol system, slightly higher values were actually achieved). Analysis of the product spectrum formed by means of headspace GC/MS was impossible in this case, since the high boilers formed have too low a vapor pressure to evaporate out of the pores of the activated carbon support in the analysis. The measured values for the different catalyst systems are collated in table 3.

TABLE 3
Overview of characteristic data of the catalyst systems
tested: ⁽¹⁾maximum conversion during hydroformylation
experiment, ⁽²⁾change in mass of the catalyst material used after
the complete experiment run time, (³ and ⁴) ratio of propanal/2-
methyl-2-pentenal or 2-methyl-2-pentanal (NK)/2-methyl-
2-pentenal by GC/MS peak areas.

| | | Xₘₐₓ/ | Δm/ | Aldehyde/ | NK/ |
|---|---|---|---|---|---|
| Catalyst | Substrate | %⁽¹⁾ | %⁽²⁾ | aldol⁽³⁾ | aldol⁽⁴⁾ |
| Rh-1/Trisopor423 | ethene | 1.0 | 2.6 | 0.3 | 0.14 |
| Rh-1/Trisopor423/ | ethene | 3.1 | −0.9 | 9.7 | 0.11 |
| C6aldol | | | | | |
| Rh-2/activated carbon | 1-butene | 0.9 | 5.2 | n.d. | n.d. |
| Rh-2/activated | 1-butene | 1.1 | 0.4 | n.d. | n.d. |
| carbon/C10aldol | | | | | |

n.d.: not determined

Very high aldol loadings are counter-productive, since it is known that elevated rhodium and ligand leaching occurs in these cases. With regard to a system having prolonged stability, a certain aldol loading should thus not be exceeded.

The experimental findings shown above can be described by our postulated model, described hereinafter, for the pore filling level.

The postulated dynamic process of pore filling with aldol condensation products and other high-boiling products is shown in FIG. 7. This is a greatly simplified illustration, since physisorption of the ligand-modified catalyst complex and/or wetting with high-boiling liquid can also take place at the support surface.

At the start of the experiment, only the exclusively physisorbed catalyst complex is present. Because of the sample preparation, at first, the rhodium catalyst complex, excess free ligand owing to the excess of ligand used and possibly residual solvent traces from the impregnation remain in and on the support material (see FIG. 7a).

After the start of the reaction, the formation of the aldehyde and associated conversion reactions such as aldol addition and condensation commence.

Because of their low volatility and the capillary pressure that exists in the porous network, the high boilers thus formed condense to a certain degree within the pores.

According to conventional pore condensation behavior, first the micropores and then the larger pores are filled. The originally physisorbed ligand-modified rhodium complex then dissolves in this condensed aldol phase and thus provides a liquid phase for the catalysis (immobilization of the ligand-modified rhodium complex) (see FIG. 7b). The reaction that thus takes place behaves like a conventional reaction in the organic solvent and has comparable n/iso selectivities. Since the proportion of the dissolved catalyst at the start of an extended experiment is low at first, the conversion is also low when macroporous supports are used. As the reaction continues, there is a gradual increase in the amount of products formed, i.e. of aldehydes, and of aldol products formed owing to further reactions, and the latter then condense in turn in the pores until an equilibrium is established between condensation and evaporation under the given reaction conditions.

From this moment on, the pore filling level remains at a constant level characteristic of a given set of reaction parameters.

In the continuous hydroformylation experiment on ethene using the macroporous Trisopor 423 support, the above-described initial phase and pore-filling phase lasts a few hours. It can be considered that a steady equilibrium state has been attained under the given reaction conditions when the pore filling reaches its equilibrium state and a defined number of pores has been filled with high-boiling components (see FIG. 7c).

In the cases in which the aldol product is explicitly already added prior to the conduct of the catalytic reaction, i.e. during the preparation, for the immobilization of the catalytically active composition (consisting of Rh complex and ligand), a particular pore filling level has already been attained on commencement of reaction. The effect of this in turn is that the dynamic process of (additional) pore filling and the establishment of the steady equilibrium state between condensation and evaporation is shortened.

This explains the lower catalyst activity in the case of Rh-1/Trisopor 423 compared to a system in which a predefined amount of aldol product (2-methyl-2-pentenal) has already been added during the catalyst preparation and accordingly prior to the conduct of the actual catalytic experiment.

### Operando DRIFTS Experiments

The catalyst material was characterized with a Bruker Vertex 80v IR spectrometer equipped by means of an additional aluminum chamber upstream of the sample space with the necessary passages to be able to evacuate the optical pathway during the measurements. DRIFTS (diffuse reflectance infrared Fourier transform spectroscopy) measurements were conducted with the “Praying Mantis” accessory and the high-temperature reaction chamber (HVC-DRP-4) from Harrick. The reaction chamber was modified with a K-type thermocouple in order to be able to measure the temperature during the reaction directly within the powder. Mass flows and pressures were adjusted using mass flow and pressure regulators from Bronkhorst. Before commencement of the reaction, the catalyst powder was heated under argon (5 ml min⁻¹, 2 bar) at 80° C. for 3 h in order to remove water and solvent residues. IR spectra were measured with a spectral resolution of 2 cm⁻¹, 151 measurements per spectrum, and a detection rate of 40 kHz. This corresponds to a measurement time of 60 s per spectrum. There were simultaneous online GC measurements with an Agilent 7890A gas chromatograph, which was used with a virtually identical setup for the analysis of the product gases in the tubular reactor system as well. The separating column used was a GS-GasPro capillary column (Agilent Technologies, length 30 m, internal diameter 0.32 mm). The oven temperature was constant at 200° C. and the measurement interval in each case was 10 min.

### Experiment in a Tubular Reactor

The catalytic study in the tubular reactor was effected in the setup described above under Catalysis experiments. The analysis system used was adopted unchanged.

1. A composition comprising:
a) at least one inert porous support material;
b) at least one metal selected from the eighth transition group of the Periodic Table of the Elements;
c) at least one organic phosphorus compound; and
d) at least one high-boiling liquid on the inert porous support material, having a lower vapour pressure than 0.074 MPa at 100° C. and 1.0 MPa,
wherein the composition is free of ionic liquids.

2. The composition as claimed in claim 1,
wherein the inert porous support material has the following material properties:
i) mean pore diameter within a range from 1 to 430 nm;
ii) pore volume within a range from 0.1 to 2 ml/g; and
iii) BET surface area within a range from 10 to 2050 m²/g.

3. The composition as claimed in claim 1,
wherein the organic phosphorus compounds are phosphines, phosphites and/or phosphoramidites.

4. The composition as claimed in claim 1, wherein the at least one organic phosphorous compound is selected from the group consisting of compounds 1, 2 and 3, wherein compounds 1, 2 and 3 comprise the following structures:

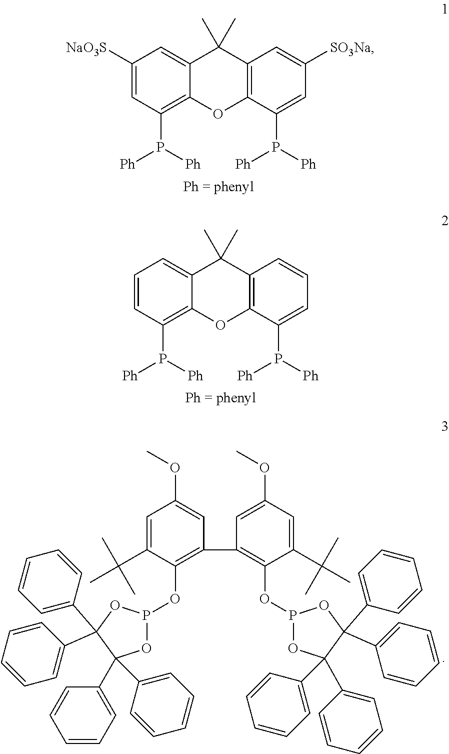

5. The composition as claimed in claim 1, wherein the high-boiling liquid is formed in situ during use in a process for chemical synthesis.

6. The composition as claimed in claim 5, wherein the high-boiling liquid is formed in situ during the hydroformylation of olefin-containing hydrocarbon mixtures.

7. A process for hydroformylating olefin-containing hydrocarbon mixtures, comprising:
a) introducing an olefin-containing hydrocarbon mixture into a reaction mixture,
b) introducing an inert porous support material into the reaction mixture,
c) introducing at least one metal selected from the group consisting of cobalt, rhodium, iridium and ruthenium into the reaction mixture,
d) introducing at least one organic phosphorus compound selected from the group consisting of phosphines, phosphites and phosphoramidites into the reaction mixture,
e) introducing an aldol compound which is not part of the olefin-containing hydrocarbon mixture into the reaction mixture,
f) feeding in H₂ and CO, and
g) heating the reaction mixture, with conversion of the olefin to an aldehyde.

8. The process as claimed in claim 7,
wherein the aldol compound in e) is at least one selected from the group consisting of:
2-methylpent-2-enal (CAS 623-36-9),
2-ethylhex-2-enal (CAS 645-62-5), and
2-propylhept-2-enal (CAS 34880-43-8).

9. The process as claimed in claim 7,
wherein the organic phosphorus compound from d) is at least one selected from the group consisting of:

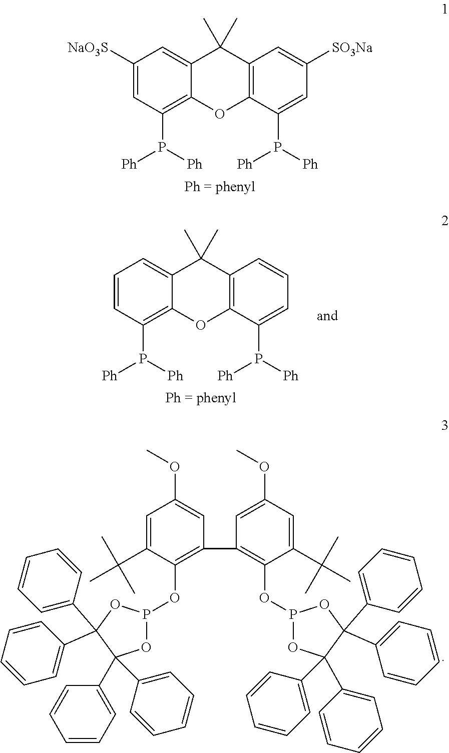

10. The process as claimed in claim 7, wherein the inert porous support material in b) has the following material properties:
i) mean pore diameter within a range from 1 to 430 nm;
ii) pore volume within a range from 0.1 to 2 ml/g; and
iii) BET surface area within a range from 10 to 2050 m²/g.

11. The process as claimed in claim 7,
wherein the olefin-containing hydrocarbon mixture is at least one selected from the group consisting of:
ethene;
propene; and
C4 olefins, C4 paraffins, polyunsaturated compounds.

12. The process as claimed in claim 7, wherein the reaction mixture is free of ionic liquids.

13. The process as claimed in claim 7, wherein the metal, the organic phosphorus compound and the aldol compound are first mixed in a separate vessel before they are introduced into the reaction vessel.

14. The process as claimed in claim 13, wherein the inert porous support material is also added to the mixture before the mixture is introduced into the reaction vessel.
